(19) United States
(12) Patent Application Publication
(10) Pub. No.: US 2019/0008750 A1
(43) Pub. Date: Jan. 10, 2019

(54) COSMETIC COMPOSITION COMPRISING A CATIONIC SURFACTANT, A FATTY ALCOHOL, AN AMPHOTERIC SURFACTANT, A STARCH DERIVATIVE, AND A CATIONIC CONDITIONING POLYMER

(71) Applicant: L'OREAL, Paris (FR)

(72) Inventors: Miriam Sayuri SAKAGUCHI, Rio de Janeiro, RJ (BR); Ana Claudia Meda SILOTO, Rio de Janeiro, RJ (BR)

(73) Assignee: L'OREAL, Paris (FR)

(21) Appl. No.: 15/752,533

(22) PCT Filed: Aug. 14, 2015
(86) PCT No.: PCT/BR2015/050119

§ 371 Date: Feb. 13, 2018

(51) Int. Cl. A61K 8/73; A61K 8/81; A61K 8/44; A61K 8/898; A61K 8/34; A61Q 5/12; A61Q 5/02

(52) U.S. Cl. CPC A61K 8/732; A61K 8/737; A61K 8/817; A61K 8/442; A61K 2800/5426; A61K 8/342; A61Q 5/12; A61Q 5/02; A61K 8/898

(57) ABSTRACT

The present invention relates to a composition for cleansing hair fibers, in particular human hair fibers, comprising (a) one or more cationic surfactants; (b) one or more amphoteric surfactants; (c) one or more fatty alcohols; (d) one or more starch derivatives; (e) one or more cationic conditioning polymers; (f) one or more amino silicones; and (g) water. These compositions impart detangling properties, curl definition, volume control, discipline, softness, conditioning, and hydrating properties to hair fibers. The invention also concerns a process for cleansing hair fibers and a use for hair care employing the composition.

### FIELD OF THE INVENTION

The present invention relates to a composition for cleansing hair fibers comprising (a) one or more cationic surfactants; (b) one or more amphoteric surfactants; (c) one or more fatty alcohols; (d) one or more starch derivatives; (e) one or more cationic conditioning polymers; (f) one or more amino silicones; and (g) water. These compositions impart detangling properties, curl definition, volume control, discipline, softness, conditioning, and hydrating properties to hair fibers.

The invention also concerns a process for cleansing and conditioning hair fibers and a use for hair care employing the composition.

### BACKGROUND OF THE INVENTION

Hair is generally damaged and embrittled by the action of external atmospheric agents such as sunlight, and also by mechanical or chemical treatments, such as brushing, combing, dyeing, bleaching, permanent-waving and/or relaxing.

Hair is thus damaged by these various factors and may over time become dry, coarse or dull, especially in fragile areas, and more particularly at the ends leading to split ends.

Cleansing compositions typically contain anionic surfactants. Anionic surfactants such as sodium lauryl sulfate have detergent properties and are highly effective at removing dirt and oil. However, anionic surfactants raise the cuticle of the hair for deep cleansing, but raised, rough cuticles also lead to frizz. Consumers with damaged, delicate, dyed, or curly hair find products containing anionic surfactants to be too drying and damaging for frequent use, and, as a result, choose mild cleansing compositions which have low levels of anionic surfactants.

However, there are drawbacks associated with mild cleansing compositions. On account of their low detergency, excess oils and styling products may not be effectively removed from the hair. This may make the hair flat and feel greasy, and with repeated use, the hair does not feel clean.

Consumers are therefore still in search of optimized cleansing compositions, for obtaining adequate visual sleekness of the hair; good feel of the hair, and in particular of wet hair; control, or even elimination, of frizziness, and also control or reduction of the volume and of the apparent mass of the head of hair, the said compositions thus being most particularly suitable for curly, damaged, and/or voluminous hair. Consumers having dyed hair are also in search of cleansing compositions which prevent color fading.

Applicants have surprisingly found that their compositions comprising (a) one or more cationic surfactants; (b) one or more amphoteric surfactants; (c) one or more fatty alcohols; (d) one or more starch derivatives; (e) one or more cationic conditioning polymers; (f) one or more amino silicones; and (g) water effectively clean the hair and provide conditioning and hydrating properties, detangling, and curl definition without leaving the hair frizzy, flat, or feeling greasy.

### SUMMARY OF THE INVENTION

The present invention is directed to compositions comprising:
(a) one or more cationic surfactants;
(b) one or more amphoteric surfactants;
(c) one or more fatty alcohols;
(d) one or more starch derivatives;
(e) one or more cationic conditioning polymers;
(f) one or more amino silicones; and
(g) water

The compositions in accordance with the invention may be used for cleansing hair fibers, for example as hair cleansers or shampoos.

The present invention is also directed to methods of applying to the hair a composition as defined above. The compositions according to the invention may be used alone or in conjunction with a hair conditioning and treatment regimen.

### DETAILED DESCRIPTION OF THE INVENTION

The present compositions provide effectively cleansing of the hair and provide conditioning and hydrating properties, detangling, and curl definition without leaving the hair frizzy, flat, or feeling greasy. When used on dyed hair, the present compositions prevent color fading.

“One or more” as used herein means at least one and thus includes individual components as well as mixtures/combinations.

“Conditioning” as used herein means imparting to one or more hair fibers at least one property chosen from combability, manageability, moisture-retentivity, luster, shine, volume control, discipline, and softness. The state of conditioning can be evaluated by any means known in the art, such as, for example, measuring, and comparing, the ease of combability of the treated hair and of the untreated hair in terms of combing work (gm-in), and consumer perception.

“Transformed hair” as used herein means hair that has undergone chemical or physical treatment to straighten or curl the hair.

“Substituted,” as used herein, means comprising one or more substituents. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Polymers,” as defined herein, include homopolymers and copolymers formed from at least two different types of monomers.

“INCI” is an abbreviation of International Nomenclature of Cosmetic Ingredients, which is a system of names provided by the International Nomenclature Committee of the Personal Care Products Council to describe personal care ingredients.

The viscosity of the composition according to the invention was measured in centipoise (cP) at 25 degrees centigrade using a using Brookfield viscometer. A person skilled in the art will be able to select the model and conditions appropriate for the sample being measured. The composition according to the invention preferably has a viscosity from about 180 to about 400 cPs, and more preferably, from about 260 to about 360 cPs.

The compositions and methods of the present invention can comprise, consist of, or consist essentially of the essential elements and limitations of the invention described herein, as well as any additional or optional ingredients, components, or limitations described herein or otherwise useful.

Other than in the operating examples, or where otherwise indicated, all numbers expressing quantities of ingredients and/or reaction conditions are to be understood as being modified in all instances by the term “about.”

All percentages, parts and ratios herein are based upon the total weight of the compositions of the present invention, unless otherwise indicated.

As used herein, all ranges provided are meant to include every specific range within, and combination of subranges between, the given ranges. Thus, a range from 1-5, includes specifically 1, 2, 3, 4 and 5, as well as subranges such as 2-5, 3-5, 2-3, 2-4, 1-4, etc.

As used herein a range of ratios is meant to include every specific ratio within, and combination of subranges between, the given ranges

In an embodiment, the present invention is directed to a composition comprising:
(a) one or more cationic surfactants;
(b) one or more amphoteric surfactants;
(c) one or more fatty alcohols;
(d) one or more starch derivatives;
(e) one or more cationic conditioning polymers; and
(f) one or more amino silicones; and
(g) water.

In one embodiment, the composition of the present invention further comprises one or more cellulosic thickening agents.

In another embodiment, the present invention is directed to a composition comprising:
(a) about 1% to about 5% by weight of one or more cationic surfactants;
(b) about 1% to about 5% by weight of one or more amphoteric surfactants;
(c) about 2% to about 11% by weight of one or more fatty alcohols;
(d) about 0.5% to about 5% by weight of one or more starch derivatives;
(e) about 0.05% to about 2% by weight of one or more cationic conditioning polymers; and
(f) about 0.1% to about 2% by weight of one or more amino silicones;
(g) about 0.1% to about 0.5% by weight of one or more cellulosic thickening agents; and
(h) about 70% to about 90% by weight of water;

all weights being relative to the total weight of the composition.

The compositions according to the invention have a soft, creamy texture that feels pleasant and is easy to apply to the hair. The composition spreads well on the hair and also rinses out easily and quickly.

The compositions according to the invention give the hair softness, sleekness and suppleness while affording a natural, healthy feel to sensitized or embrittled hair. The compositions according to the invention are particularly suitable for use on all types of hair such as curly, sensitized, straight, or colored/dyed hair, and enables shiny and supple curls to be obtained.

Another subject of the present invention is a process for treating hair fibers, such as the hair, which comprises applying a composition as described above on the fibers in an amount effective to cleanse the hair.

The compositions according to the invention improve the cosmetic properties of hair fibers, in particular human hair fibers such as the hair, for example, in terms of softness, smoothness and/or suppleness while at same time giving the hair a natural look and feeling.

The present invention also relates to a process for treating the hair, comprising the application to the fibers of the composition according to the invention.

The invention also relates to the use of the composition according to the invention as a hair cleanser, preferably in place of a shampoo and conditioner.

The compositions described above may be used on any type of hair, for example, light or dark hair, straight or curly, natural hair, or hair that has undergone a cosmetic treatment such as permanent waving, dyeing, bleaching or relaxing.

In a preferred embodiment, the composition of the present invention is applied on curly, sensitized, embrittled, and/or damaged hair.

The application to the hair of the composition according to the invention may be performed, for example, using a comb, a fine brush, a coarse brush or with the fingers.

The invention also relates to a process for making the composition according to the invention, comprising combining (a) one or more cationic surfactants; (b) one or more amphoteric surfactants; (c) one or more fatty alcohols; (d) one or more starch derivatives; (e) one or more cationic conditioning polymers; (f) one or more amino silicones; and (g) water.

Other subjects and characteristics, aspects and advantages of the invention will emerge even more clearly on reading the description and the example that follows.

### Cationic Surfactant

The composition according to the invention comprises one or more cationic surfactants.

Non-limiting examples of cationic surfactants useful in the invention include, for example, optionally polyoxyalkylenated primary, secondary or tertiary fatty amine salts, quaternary ammonium salts, and mixtures thereof.

Quaternary ammonium salts useful in the invention include, for example:
quaternary ammonium salts having formula (Ia):

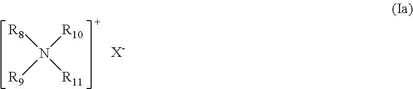

in which:

the groups R8 to R11, which may be identical or different, represent a linear or branched aliphatic group containing from 1 to 30 carbon atoms, or an aromatic group such as aryl or alkylaryl, at least one of the groups R8 to R11 containing from 8 to 30 carbon atoms and preferably from 12 to 24 carbon atoms; it being possible for the aliphatic groups to comprise heteroatoms such as, in particular, oxygen, nitrogen, sulfur or halogens;

The aliphatic groups are chosen, for example, from C1-C30 alkyl, C1-C30 alkoxy, (C2-C6)polyoxyalkylene, C1-C30 alkylamide, (C12-C22)alkyl(C2C6)alkylamido, (C12-C22)alkyl acetate and C1-C30 hydroxyalkyl groups.

X— is an anion chosen from the group consisting of halides, phosphates, acetates, lactates, (C1-C4)alkyl sulfates, (C1-C4)alkylsulfonates and (C1-C4)alkylarylsulfonates.

Among the quaternary ammonium salts having formula (Ia), preference is given to tetraalkylammonium chlorides such as, for example, dialkyldimethylammonium or alkyltrimethylammonium chlorides in which the alkyl group comprises approximately from 12 to 22 carbon atoms, particularly behenyltrimethylammonium, distearyldimethylammonium, cetyltrimethylammonium, and benzyldimethylstearylammonium chlorides, or secondly, to palmitylamidopropyltrimethylammonium chloride or stearamidopropyldimethyl(myristyl acetate)-ammonium chloride, which is sold under the name Ceraphyl® 70 by the company Van Dyk.
imidazoline quaternary ammonium salts having formula (IIa):

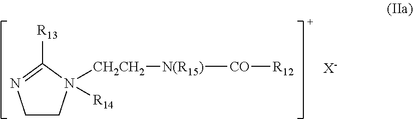

in which

R12 represents an alkenyl or alkyl group comprising from 8 to 30 carbon atoms, for example fatty acid derivatives of tallow;

R13 represents a hydrogen atom, a C1-C4 alkyl group or an alkenyl or alkyl group comprising from 8 to 30 carbon atoms;

R14 represents a C1-C4 alkyl group;

R15 represents a hydrogen atom or a C1-C4 alkyl group;

X— is an anion chosen from the group consisting of halides, phosphates, acetates, lactates, (C1-C4)alkyl sulfates, (C1-C4)alkylsulfonates and (C1-C4)alkylarylsulfonates;

R12 and R13 preferably denote a mixture of alkenyl or alkyl groups containing from 12 to 21 carbon atoms, for example fatty acid derivatives of tallow, R14 denotes a methyl group, and R15 denotes a hydrogen atom. A product of this kind is sold for example under the name Rewoquat® W 75 by the company Rewo.
quaternary di- or triammonium salts having formula (IIIa):

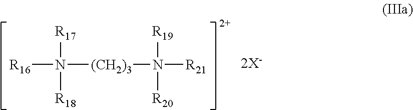

in which

R16 denotes an alkyl group containing approximately from 16 to 30 carbon atoms, which is optionally hydroxylated and/or interrupted with one or more oxygen atoms, R17 is chosen from hydrogen or an alkyl group containing from 1 to 4 carbon atoms or a group —(CH2)3—N+(R16a)(R17a)(R18a);

R16a, R17a, R18a, R18, R19, R20 and R21, which may be identical or different, are chosen from hydrogen and an alkyl group comprising from 1 to 4 carbon atoms; and

X— is an anion chosen from the group consisting of halides, acetates, phosphates, nitrates, (C1-C4)alkyl sulfates, (C1-C4)alkylsulfonates and (C1-C4)alkylarylsulfonates, in particular methyl sulfate and ethyl sulfate.

Such compounds are, for example, Finquat® CT-P, sold by the company Finetex (Quaternium 89), and Finquat® CT, sold by the company Finetex (Quaternium 75),
quaternary ammonium salts containing one or more ester functions having the following formula (IVa):

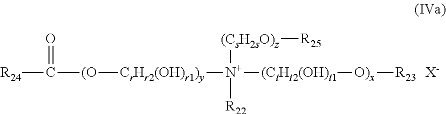

in which:

R22 is chosen from C1-C6 alkyl groups and C1-C6 hydroxyalkyl or C1-C6 dihydroxyalkyl groups;

R23 is chosen from the group R26—C(═O)—; hydrocarbon-based linear or branched, saturated or unsaturated C1-C22 groups R27; and a hydrogen atom;

R25 is chosen from the group R28—C(═O)—; hydrocarbon-based linear or branched, saturated or unsaturated C1-C6 groups R29; and a hydrogen atom;

R24, R26 and R28, which may be identical or different, are chosen from linear or branched, saturated or unsaturated C7-C21 hydrocarbon-based groups;

r, s and t, which may be identical or different, are integers ranging from 2 to 6;

r1 and t1, which may be identical or different, are equal to 0 or 1;

r2+r1=2 r and t1+t2=2 t;

y is an integer ranging from 1 to 10;

x and z, which may be identical or different, are integers ranging from 0 to 10;

X— is a simple or complex, organic or mineral anion;

with the proviso that the sum x+y+z is from 1 to 15, that when x is 0 then R23 denotes R27, and that when z is 0 then R25 denotes R29.

The alkyl groups R22 may be linear or branched, and more particularly linear. Preferably, R22 denotes a methyl, ethyl, hydroxyethyl or dihydroxypropyl group, and more particularly a methyl or ethyl group.

Advantageously, the sum x+y+z is from 1 to 10.

When R23 is an R27 hydrocarbon group, it may be long and may have from 12 to 22 carbon atoms, or may be short and may have from 1 to 3 carbon atoms.

When R25 is an R29 hydrocarbon group, it preferably has 1 to 3 carbon atoms.

Advantageously, R24, R26 and R28, which are identical or different, are chosen from linear or branched, saturated or unsaturated C11-C21 hydrocarbon groups, and more particularly from linear or branched, saturated or unsaturated C11-C21 alkyl and alkenyl groups.

Preferably, x and z, which may be identical or different, are equal to 0 or 1. Advantageously, y is equal to 1. Preferably, r, s and t, which may be identical or different, are equal to 2 or 3, and even more particularly are equal to 2.

The anion X— is preferably a halide, preferably chloride, bromide or iodide, a (C1-C4)alkyl sulfate, (C1-C4)alkyl sulfonate or (C1-C4)alkylaryl sulfonate. However, it is possible to use methanesulfonate, phosphate, nitrate, tosylate, an anion derived from an organic acid, such as acetate or lactate, or any other anion that is compatible with the ammonium comprising an ester function.

The anion X— is more particularly still chloride, methyl sulfate or ethyl sulfate.

Use is made more particularly, in the composition according to the invention, of the ammonium salts having formula (IVb) in which:
R22 denotes a methyl or ethyl group,
x and y are equal to 1,
z is equal to 0 or 1,
r, s and t are equal to 2,
R23 is chosen from the group R26—C(═O)—; methyl groups, ethyl groups or hydrocarbon-based C14-C22 groups; and a hydrogen atom,
R25 is chosen from the group R28—C(═O)—; and a hydrogen atom,
R24, R26 and R28, which may be identical or different, are chosen from linear or branched, saturated or unsaturated C13-C17 hydrocarbon groups, and preferably from linear or branched, saturated or unsaturated C13-C17 alkyl and alkenyl groups.

The hydrocarbon-based groups are advantageously linear.

Among the compounds of formula (IVb), examples that may be mentioned include salts, in particular the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent, such as an alkyl halide, preferably methyl or ethyl halide, a dialkyl sulfate, preferably methyl or ethyl sulfate, methyl methanesulfonate, methyl para-toluenesulfonate, glycol chlorohydrin or glycerol chlorohydrin. Such compounds are, for example, sold under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a weight majority of diester salts. Use may also be made of the ammonium salts containing at least one ester function that are described in U.S. Pat. No. 4,874,554 and U.S. Pat. No. 4,137,180. Use may also be made of behenoylhydroxypropyltrimethylammonium chloride, for example, sold by the company Kao under the name Quartamin BTC 131.

Preferably, the ammonium salts containing at least one ester function.

Preferably, the cationic surfactants are chosen from cetyltrimethylammonium, behenyltrimethylammonium, and dipalmitoylethylhydroxyethylmethylammonium salts, and mixtures thereof. Most preferably, the cationic surfactant is known under the INCI name behentrimonim chloride.

The composition according to the invention comprise the cationic surfactant in an amount ranging from about 0.1% to about 10% by weight, preferably from about 0.5% to about 8% by weight, and most preferably from about 1% to about 5% by weight, relative to the total weight of the composition.

### Amphoteric Surfactant

The composition according to the invention comprises an amphoteric surfactant.

Non-limiting examples of amphoteric surfactants useful in the invention include, for example, optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C8-C20)alkylbetaines, sulfobetaines, (C8-C20)alkylsulfobetaines, (C8-C20)alkylamido(C1-C6)alkylbetaines, such as cocamidopropylbetaine, and (C8-C20)alkylamido(C1-C6)alkylsulfobetaines, and mixtures thereof.

Among the optionally quaternized secondary or tertiary aliphatic amine derivatives that may be used, mention may also be made of the products of respective structures (A1) and (A2) below:

Rₐ—CON(Z)CH₂₋(CH₂)ₘ—N⁺(R_{b})(R_{c})(CH₂COO⁻) (A1)

in which:

Rₐ represents a C₁₀-C₃₀ alkyl or alkenyl group derived from an acid Rₐ—COOH preferably present in hydrolysed coconut oil, a heptyl group, a nonyl group or an undecyl group,

R_{b} represents a β-hydroxyethyl group,

R_{c} represents a carboxymethyl group;

m is equal to 0, 1 or 2,

Z represents a hydrogen atom or a hydroxyethyl or carboxymethyl group;

R_{a′}—CON(Z)CH₂₋(CH₂)_{m′}—N(B)(B′) (A2)

in which:

B represents —CH₂CH₂OX′, with X′ representing —CH₂—COOH, CH₂—COOZ′, —CH₂CH₂—COOH, —CH₂CH₂—COOZ′, or a hydrogen atom,

B′ represents —(CH₂)_{z}—Y′, with z=1 or 2, and Y′ representing —COOH, —COOZ′, —CH₂—CHOH—SO₃H or —CH₂—CHO—SO₃Z′,

m′ is equal to 0, 1 or 2,

Z represents a hydrogen atom or a hydroxyethyl or carboxymethyl group,

Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane,

R_{a′} represents a C₁₀-C₃₀ alkyl or alkenyl group of an acid R_{a′}COOH preferably present in hydrolysed linseed oil or coconut oil, an alkyl group, in particular a C₁₇ alkyl group, and its iso form, or an unsaturated C₁₇ group.

Among the compounds corresponding to formula (A2) in which X′ represents an hydrogen atom, mention may be made of compounds classified under the INCI names sodium cocoamphoacetate, sodium lauroamphoacetate, sodium caproamphoacetate and sodium capryloamphoacetate.

Other compounds corresponding to formula (A2) are disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

Examples that may be mentioned include the cocoamphodiacetate sold by the company Rhodia under the trade name Miranol® C2M Concentrate, the sodium cocoamphoacetate sold under the trade name Miranol Ultra C 32 and the product sold by the company Chimex under the trade name CHIMEXANE HA.

Preferably, the amphoteric surfactants are chosen from (C8-C20)alkylbetaines such as the one known under the INCI names coco-betaine, (C8-C20)alkylamido(C1-C6)alkylbetaines such as the one known under the INCI name cocamidopropylbetaine, and mixtures thereof. Even more preferentially, the amphoteric surfactant is coco-betaine.

The composition according to the invention comprises the amphoteric surfactant(s) in an amount ranging from about 0.1% to about 10% by weight, preferably from about 0.5% to about 8% by weight, and most preferentially from about 1% to about 5% by weight relative to the total weight of the composition.

### Fatty Alcohol

The composition according to the invention comprises one or more fatty alcohols comprising 8 to 30 carbon atoms and may be saturated or unsaturated.

The fatty alcohols preferably correspond to the formula R-OH in which R is a saturated or unsaturated, linear or branched hydrocarbon-based radical, comprising 8 to 30 carbon atoms, optionally comprising one or more OH groups.

Preferably, R comprises from 10 to 22 carbon atoms, or even from 12 to 20 carbon atoms.

Preferably, R is a saturated, linear or branched radical.

The saturated fatty alcohols are preferably branched. They can optionally comprise, in their structure, at least one aromatic or non-aromatic ring. They are preferably acyclic. Among liquid saturated fatty alcohols, octyldodecanol, isostearyl alcohol and 2-hexyldecanol can be cited.

The unsaturated fatty alcohols exhibit, in their structure, at least one double or triple bond and preferably one or more double bonds. When several double bonds are present, there are preferably 2 or 3 of them and they can be conjugated or unconjugated. These unsaturated fatty alcohols can be linear or branched. They can optionally comprise, in their structure, at least one aromatic or non-aromatic ring. They are preferably acyclic. Among the liquid unsaturated fatty alcohols, oleyl alcohol, linoleyl alcohol, linolenyl alcohol and undecylenyl alcohol may be mentioned.

Preferably, the composition comprises one or more saturated linear fatty alcohols, comprising 8 to 30 carbon atoms, chosen from myristyl alcohol, cetyl alcohol, stearyl alcohol, cetearyl alcohol, and mixtures thereof. Most preferably, the fatty alcohol is cetearyl alcohol.

The composition according to the invention comprises the fatty alcohol in an amount ranging from about 1% to about 20% by weight, preferably from about 2% to about 15% by weight, and most preferably from about 4% to about 10% by weight, relative to the total weight of the composition.

### Starch Derivative

The cosmetic composition according to the invention comprises a starch derivative chosen from the compounds of the following formulae:

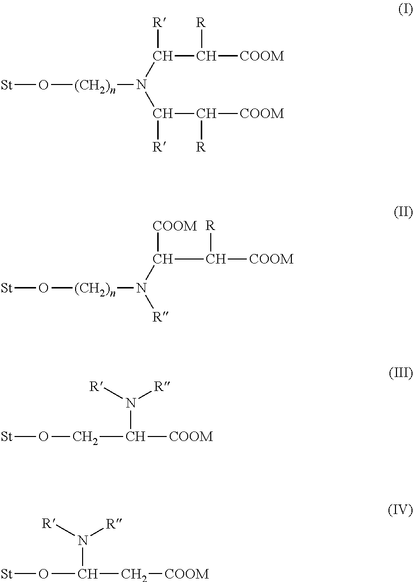

in which formulae:

St-O represents a starch molecule,

R, which may be identical or different, represents a hydrogen atom or a methyl radical,

R′, which may be identical or different, represents a hydrogen atom, a methyl radical or a —COOH group,

n is an integer equal to 2 or 3,

M, which may be identical or different, denotes a hydrogen atom, an alkali metal or alkaline-earth metal such as Na, K or Li, NH₄, a quaternary ammonium or an organic amine,

R″ represents a hydrogen atom or an alkyl radical containing from 1 to 18 carbon atoms.

These compounds are disclosed in particular in U.S. Pat. Nos. 5,455,340 and 4,017,460 which are included by way of reference.

The starch molecules may be derived from any plant sources of starch such as, in particular, corn, potato, oat, rice, tapioca, sorghum, barley or wheat. The starch hydrolysates mentioned above may also be used. The starch is preferably derived from potato.

The starches of formula (I) or (II) are used in particular. Starches modified with 2-chloroethylaminodipropionic acid, i.e. the starches of formula (I) or (II) in which R, R′ and R″ represent a hydrogen atom and n is equal to 2, are used more preferentially.

A preferred starch derivative is known under the INCI name potato starch modified sold under the tradename STRUCTURE® SOLANACE by AkzoNobel.

The composition according to the invention comprises the starch derivative in an amount ranging from about 0.1% to about 10%, preferably from about 0.5% to about 8%, and most preferably from about 0.5% to about 5% by weight relative to the total weight of the composition.

### Cationic Conditioning Polymer

The composition according to the invention comprises one or more cationic polymers. The cationic conditioning polymer used in the invention comprises homopolymers, copolymers, and mixtures thereof.

Non-limiting examples of cationic conditioning polymers useful in the invention include, for example, cationic cellulose derivatives, such as for example polyquaternium-10; cationic gum derivatives such as for example gum derivatives, including particularly guar hydroxypropyltrimonium chloride; polymer derivatives of diallyldimethyl ammonium chloride (“poly-DADMAs”) and of methacrylamidopropyltrimethylammonium chloride (“poly-MAPTACs”), and mixtures thereof

Non-limiting examples of poly-DADMAs and poly- poly-MAPTACs include, polyquaternium-4, polyquaternium-5, polyquaternium-6, polyquaternium-7, polyquaternium-22, polyquaternium-37, polyquaternium-39, polyquaternium-47, polyquaternium-53, and mixtures thereof.

The composition according to the invention preferably comprises cationic polymers known under the INCI names polyquaternium-7, guar hydroxypropyltrimonium chloride, and mixtures thereof.

The cationic polymer is in an amount ranging from about 0.01% to about 4% by weight, preferably from about 0.05% to about 3% by weight, and most preferably from about 0.05% to about 2% by weight, relative to the total weight of the composition.

### Amino Silicones

The cosmetic composition according to the invention optionally may comprise one or more amino silicones. The term “amino silicone” is intended to mean any silicone comprising at least one primary, secondary or tertiary amine or a quaternary ammonium group.

As amino silicone that may be used in the scope of the invention, the following can be cited:

a) polysiloxanes corresponding to formula (A):

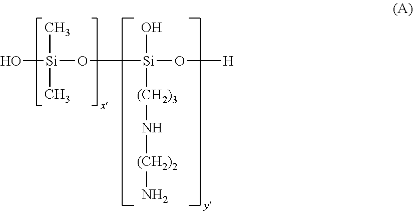

in which x′ and y′ are integers such that the weight-average molecular weight (Mw) is comprised between about 5000 and 500 000;

b) amino silicones corresponding to formula (B):

R′aG3-a-Si(OSiG2)n-(OSiGbR′2-b)m-O-SiG3-a-R′a (B)

in which:
G, which may be identical or different, designate a hydrogen atom, or a phenyl, OH or C1-C8 alkyl group, for example methyl, or C1-C8 alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula -CqH2qL in which q is a number ranging from 2 to 8 and L is an optionally quaternized amino group chosen from the following groups:

—NR″-Q-N(R″)2

—N(R″)2

—N+(R″)3 A-

—N+H(R″)2 A-

—N+H2(R″) A-

—N(R″)-Q-N+R″H2 A-

—NR″-Q- N+(R″)2H A-

—NR″-Q-N+(R″)3 A-,

in which R″, which may be identical or different, denote hydrogen, phenyl, benzyl, or a saturated monovalent hydrocarbon-based radical, for example a C1-C20 alkyl radical; Q denotes a linear or branched CrH2r group, r being an integer ranging from 2 to 6, preferably from 2 to 4; and A- represents a cosmetically acceptable ion, in particular a halide such as fluoride, chloride, bromide or iodide.

A group of amino silicones corresponding to this definition (B) is represented by the silicones called “trimethylsilylamodimethicone” having formula (C).

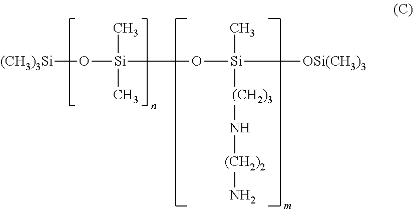

in which n and m have the meanings given above, in formula B.

Another group of amino silicones corresponding to this definition is represented by silicones having the following formulae (D) or (E):

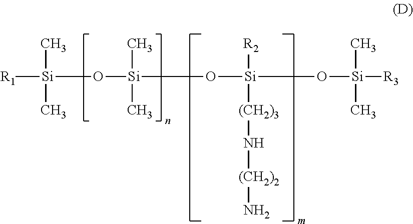

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R1, R2, R3, which may be identical or different, represent a hydroxy or C1-C4 alkoxy radical, where at least one of the radicals R1 to R3 denotes an alkoxy radical.

The alkoxy radical is preferably a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular weight (Mw) of the silicone ranges preferably from 2000 to 1 000 000, more particularly from 3500 to 200 000.

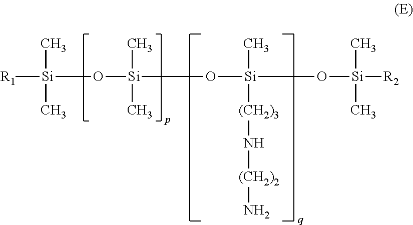

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R1, R2, which are different, represent a hydroxy or C1-C4 alkoxy radical, where at least one of the radicals R1 or R2 denotes an alkoxy radical.

The alkoxy radical is preferably a methoxy radical.

The hydroxy/alkoxy mole ratio ranges generally from 1:0.8 to 1:1.1 and preferably from 1:0.9 to 1:1 and more particularly equals 1:0.95.

The weight-average molecular weight (Mw) of the silicone ranges preferably from 2000 to 200 000, even more particularly 5000 to 100 000 and more particularly from 10 000 to 50 000.

Commercial products corresponding to these silicones having structure (D) or (E) may include in their composition one or more other amino silicones whose structure is different than formulae (D) or (E).

A product containing amino silicones having structure (D) is sold by Wacker under the name Belsil® ADM 652.

A product containing amino silicones having structure (E) is sold by Wacker under the name Fluid WR 1300e.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometres. Preferably, in particular as amino silicones having formula (E), microemulsions are used whose average particle size ranges from 5 nm to 60 nanometres (limits included) and more preferably from 10 nm to 50 nanometres (limits included). Accordingly, according to the invention the microemulsions of amino silicone having formula (E) sold as Finish CT 96 E® or SLM 28020® by Wacker can be used.

Another group of amino silicones corresponding to this definition is represented by the following formula (F):

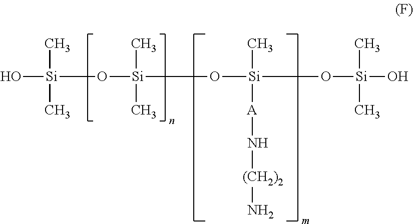

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

A preferred silicone of formula (F) is amodimethicone (INCI name) sold under the tradename XIAMETER® MEM-8299 Cationic Emulsion by Dow Corning.

Another group of amino silicones corresponding to this definition is represented by the following formula (G):

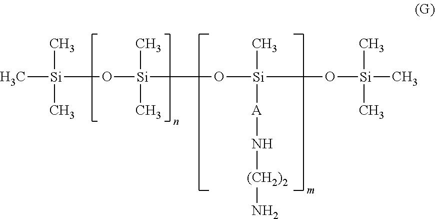

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

A silicone having this formula is for example DC2-8566 Amino Fluid by Dow Corning.

c) amino silicones corresponding to formula (H):

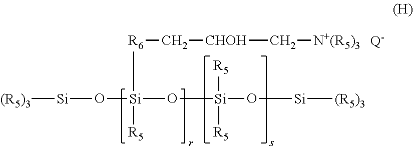

in which:
R5 represents a monovalent hydrocarbon-based radical containing from 1 to 18 carbon atoms, and in particular a C1-C18 alkyl or C2-C18 alkenyl radical, for example methyl;
R6 represents a divalent hydrocarbon-based radical, in particular a C1-C18 alkylene radical or a divalent C1-C18, for example C₁-C8, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in patent U.S. Pat. No. 4,185,087.

d) quaternary ammonium silicones having formula (I):

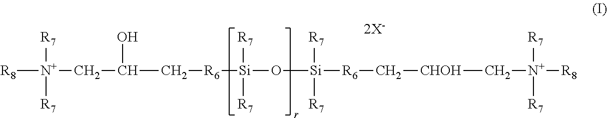

in which:
R7, which may be identical or different, represent a monovalent hydrocarbon-based radical containing from 1 to 18 carbon atoms, and in particular a C1-C18 alkyl radical, a C2-C18 alkenyl radical or a ring containing 5 or 6 carbon atoms, for example methyl;
R6 represents a divalent hydrocarbon-based radical, in particular a C1-C18 alkylene radical or a divalent C1-C18, for example C₁-C8, alkylenoxy radical linked to the Si via an SiC bond;
R8, which may be identical or different, represent a hydrogen atom, a monovalent hydrocarbon-based radical containing from 1 to 18 carbon atoms, and in particular a C1-C18 alkyl radical, a C2-C18 alkenyl radical or a —R6—NHCOR7 radical;
X— is an anion such as a halide ion, in particular chloride, or an organic acid salt (for example acetate);
r represents a mean statistical value from 2 to 200 and in particular from 5 to 100;

These silicones are described, for example, in patent application EP-A 0 530 974.

e) amino silicones having formula (J):

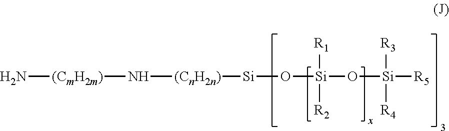

in which:
R1, R2, R3 and R4, which may be identical or different, denote a C1-C4 alkyl radical or a phenyl group;
R5 denotes a C1-C4 alkyl radical or a hydroxyl group;
n is an integer ranging from 1 to 5;
m is an integer ranging from 1 to 5;
   and in which x is chosen such that the amine number is between 0.01 and 1 meq/g;

f) multiblock polyoxyalkylenated amino silicones, of type (AB)n, A being a polysiloxane block and B being a polyoxyalkylenated block containing at least one amine group.

Said silicones are preferably constituted of repeating units having the following general formulae:

[—(SiMe2O)xSiMe2—R—N(R″)—R′—O(C2H4O)a(C3H6O)b—R′—N(H)—R—]

or alternatively

[—(SiMe2O)xSiMe2—R—N(R″)—R′—O(C2H4O)a(C3H6O)b—]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C2-C12 hydrocarbon-based radical, optionally including one or more heteroatoms such as oxygen; preferably, R denotes an ethylene radical, a linear or branched propylene radical, a linear or branched butylene radical, or a —CH2CH2CH2OCH(OH)CH2— radical; preferentially R denotes a —CH2CH2CH2OCH(OH)CH2— radical;
R′, which may be identical or different, represent a divalent linear or branched C2-C12 hydrocarbon-based radical, optionally including one or more heteroatoms such as oxygen; preferably, R′ denotes an ethylene radical, a linear or branched propylene radical, a linear or branched butylene radical, or a —CH2CH2CH2OCH(OH)CH2— radical; preferentially R′ denotes —CH(CH3)—CH2—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably comprised between 5000 and 1 000 000, more particularly between 10 000 and 200 000.

Mention may be made especially of the silicones sold under the names Silsoft™ A-843 or Silsoft™ A+ by Momentive.

g) the alkylamino silicones corresponding to formula (K) below:

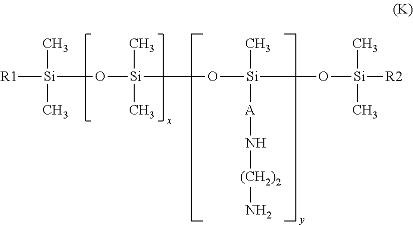

in which:
x and y are numbers ranging from 1 to 5000; preferably, x ranges from 10 to 2000 and especially from 100 to 1000; preferably, y ranges from 1 to 100;
R1 and R2, which may be identical or different, preferably identical, are linear or branched, saturated or unsaturated alkyl radicals, comprising 6 to 30 carbon atoms, preferably 8 to 24 carbon atoms and especially 12 to 20 carbon atoms;
A denotes a linear or branched alkylene radical containing from 2 to 8 carbon atoms,

Preferably, A comprises 3 to 6 carbon atoms, especially 4 carbon atoms; preferably, A is branched. Mention may be made especially of the following divalent radicals: —CH2CH2CH2 and —CH2CH(CH3)CH2—.

Preferably, R1 and R2, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R1 and R2, which may be identical or different, are chosen from hexadecyl (cetyl) and octadecyl (stearyl) radicals.

Preferentially, the silicone is of formula (K) with:
x ranging from 10 to 2000 and especially from 100 to 1000;
y ranging from 1 to 100;
A comprising 3 to 6 carbon atoms and especially 4 carbon atoms; preferably, A is branched; and more particularly A is chosen from the following divalent radicals: —CH2CH2CH2 and —CH2CH(CH3)CH2—; and
R1 and R2, which may be identical or different, being linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R1 and R2, which may be identical or different, being chosen from hexadecyl (cetyl) and octadecyl (stearyl) radicals.

A preferred silicone of formula (K) is bis-cetearyl amodimethicone (INCI name).

Mention may be made especially of the silicone sold under the name Silsoft™ AX by Momentive.

Preferably, the amino silicones according to the invention are chosen from the amino silicones of formula (F). A preferred silicone of formula (F) is amodimethicone (INCI name) sold under the tradename XIAMETER® MEM-8299 Cationic Emulsion by Dow Corning.

When present in the composition according to the invention, the amino silicone is in an amount ranging from about 0.01% to about 5% by weight, preferably from about 0.05% to about 3% by weight, and most preferably from about 0.1% to about 1% by weight, relative to the total weight of the composition.

### Water

The composition according to the invention comprises water. The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

The composition according to the invention comprises water in an amount ranging from about 50% to about 99% by weight, preferably from about 65% to about 95% by weight, and most preferably from about 70% to about 92% by weight, relative to the total weight of the composition.

### Cellulosic Thickening Agents (Optional)

The composition according to the invention optionally may comprise one or more cellulosic thickening agents.

Non-limiting examples of cellulosic thickening agents useful in the invention include, for example, cellulose and modified cellulosic compositions such as, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers some portion of the hydroxy groups of the cellulose polymer are hydroyxalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof.

When present in the composition according to the invention, the cellulosic thickening agent preferably comprises an alkyl hydroxyalkyl cellulose ether thickening agent known under the INCI name cetyl hydroxyethylcellulose, which is the ether of cetyl alcohol and hydroxyethylcellulose. This material is sold under the tradename NATROSOL™ Plus 330 CS from Ashland.

When present in the composition according to the invention, the cellulosic thickening agent is preferably in an amount ranging from about 0.01% to about 2% by weight, preferably from about 0.05% to about 1% by weight, and most preferably from about 0.1% to about 0.6% by weight, relative to the total weight of the composition.

### Water-Soluble Solvent (Optional)

The compositions according to the invention optionally may comprise one or more water-soluble solvent. The term “water-soluble solvent” means a compound that is liquid at room temperature and water-miscible (miscibility with water of greater than 50% by weight at 25° C. and atmospheric pressure).

Non-limiting examples of water-soluble solvents useful in the invention include, for example, glycols containing from 2 to 8 carbon atoms, such as ethylene glycol, propylene glycol, 1,3-butylene glycol, hexylene glycol, and dipropylene glycol, glycerin, and mixtures thereof.

When present in the composition according to the invention, the water-soluble solvent is preferably present in the composition according to the invention in an amount of from about 0.01% to 10% by weight, preferably in an amount of from about 0.05% to 5% by weight, and most preferably 0.1% to 1% by weight, based on the total weight of the composition.

The composition according to the invention may also comprise one or more standard additives that are well known in the art, chosen from fragrances, preservatives, pH adjusting agents, colorants, chelating agents, antioxidants, moisturizers, emollients, amino acids, proteins, biological polymers, active agents, ceramides or pseudoceramides, oils and waxes, vitamins or provitamins, and mixtures thereof.

A person skilled in the art will take care to select the optional standard additives and the amount thereof such that they do not harm the properties of the compositions of the present invention.

These standard additives are generally present in the composition according to the invention in an amount ranging from about 0 to about 20% by weight relative to the total weight of the composition.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

The compositions according to the invention can be manufactured by known processes used generally in the cosmetics, including the processes described in the examples below.

The examples below are given as illustrations of the present invention.

### EXAMPLES

TABLE 1
Inventive Examples

| | | % by Weight | |
|---|---|---|---|
| | | (Active Raw Material) | |
| PHASE | INCI NAME | Example 1 | Example 2 |
| A | GUAR | 0.12 | 0.12 |
| | HYDROXYPROPYLTRIMONIUM | | |
| | CHLORIDE | | |
| B | CETEARYL ALCOHOL | 7 | 7 |
| B | POTATO STARCH MODIFIED | 1.03 | 1.03 |
| | (STRUCTURE ® SOLANACE from | | |
| | AkzoNobel) | | |
| B | CETYL | 0 | 0.23 |
| | HYDROXYETHYLCELLULOSE | | |
| | (NATROSOL ™ Plus 330 CS from | | |
| | Ashland) | | |
| C | COCO-BETAINE | 2.1 | 2.1 |
| C | CITRIC ACID | 0.02 | 0.02 |
| D | BEHENTRIMONIUM CHLORIDE | 2.37 | 2.37 |
| D | GLYCERIN | 0.2 | 0.2 |
| E | AMODIMETHICONE | 0.58 | 0.58 |
| | (XIAMETER ® MEM-8299 Cationic | | |
| | Emulsion from Dow Corning) | | |
| E | CETRIMONIUM CHLORIDE | 0.01 | 0.01 |
| E | TRIDECETH-6 | 0.05 | 0.05 |
| E | POLYQUATERNIUM-7 | 0.36 | 0.36 |
| F | PRESERVATIVES | 0.5 | 0.5 |
| F | FRAGRANCE | 0.6 | 0.8 |
| | WATER | q.s. | q.s. |
| | TOTAL | 100% | 100% |

In making each of the examples in Table 1, the following procedure was used. In a side kettle, phase A was solubilized in a small amount of water. The ingredients of phase B were added to the main kettle with a portion of water and mixed at 80° C. The contents of the side kettle were added to the main kettle followed by mixing. The ingredients of phase C were added to the main kettle with mixing. The temperature of the main kettle was cooled to 70° C., and the ingredients of phase D were added followed by mixing. An additional portion of water was added to the main kettle, and the temperature was lowered to 45° C. with mixing. The ingredients of phase E were added to the main kettle followed by mixing. The contents of the main kettle were allowed to cool below 45° C. followed by addition of the remainder of the water and he ingredients of phase F. Mixing was continued until the batch was homogeneous.

TABLE 2
Comparative Example

| | % by Weight |
|---|---|
| | (Active Raw Material) |
| INCI NAME | Example 3 |
| CETEARYL ALCOHOL | 7 |
| HYDROXYPROPYL STARCH PHOSPHATE | 3.5 |
| SODIUM COCOAMPHOPROPIONATE | 5 |
| BEHENTRIMONIUM CHLORIDE | 3.16 |
| AMODIMETHICONE | 1.0 |
| (XIAMETER ® MEM-8299 Cationic | |
| Emulsion from Dow Corning) | |
| CETRIMONIUM CHLORIDE | 0.02 |
| TRIDECETH-6 | 0.09 |
| PRESERVATIVES | 0.6 |
| FRAGRANCE | 0.8 |
| WATER | q.s. |
| TOTAL | 100% |

TABLE 3
Summary of Qualitative Evaluation
Results of Example 2 (Inventive) vs. Example 3 (Comparative)

| | | Liking Rating |
|---|---|---|
| | | (Inventive vs. |
| Attribute | Hair Type* | Comparative) |
| Frizz control | I to V | Inventive |
| Hydration on dry hair | I to V | Inventive |
| Ease of combing wet hair | I to V | Inventive |
| Overall liking | I (straight) | Inventive |
| Softness | I (straight) | Inventive |
| Detangling | I (straight) | Inventive |
| Shine | I (straight) | Inventive |
| Reduced hair breakage | I to V | Inventive |
| Softness | I to V Dyed | Inventive |
| Moisturizing | I to V Dyed | Inventive |
| Prevents color fading | I to V Dyed | Inventive |
| Lightness | I to V Dyed | Inventive |
| Reduced hair breakage/loss | III not transformed | Comparative |
| | (curly) | |
| Hair left moisturized | III not transformed | Comparative |
| | (curly) | |
| Protection from heat of flat | III not transformed | Comparative |
| iron or blow dry | (curly) | |
| Keeps hair clean throughout | III not transformed | Comparative |
| the day | (curly) | |
| Does not make hair look dirty | III not transformed | Comparative |
| | (curly) | |
| Texture/consistency of hair | III not transformed | Comparative |
| | (curly) | |

*Hair Type I: Straight Caucasian; Type II: Caucasian with slight wavy;
Type III: Curly Caucasian; Type IV: Highly Curly Caucasian; Type V: Coily African.

A quantitative (monadic) test was conducted to obtain consumers' opinion, perceptions and overall satisfaction with Example 2 compared to Example 3. The test panel contained 800 women having hair types I to V who are regular users of a shampoo and conditioner regimen. Hair types were further separated by transformed and not transformed hair. Panelists participated in a blinded home evaluation for 15 days. Panelists were instructed to use the test product as they normally would in place of their usual shampoo and conditioner regimen for 7 days. After 7 days of use, panelists responded to a semi-qualitative questionnaire with closed questions. The results are summarized in Table 3.

For overall liking ratings and most benefits of the inventive composition, Example 2, were found to be on par with the comparative example, Example 3. In addition, overall, Example 2 performed better than Example 3 in the areas of frizz control, hydration on dry hair, ease of combing wet hair, reduced hair breakage. Also, Example 2 performed better than Example 3 in the areas of softness, moisturizing, prevents color fading and lightness for types I to V dyed hair. For other attributes such as keeps hair clean throughout the day, reduced hair breakage/loss, left hair moisturized, protection from the heat of flat iron or blow-dry, does not make your hair look dirty quickly, and texture/consistency (tending to thick), the panel preferred Example 3 over Example 2 only when tested on hair type III (not transformed).

1.-16. (canceled)

17. A composition comprising:
(a) at least one cationic surfactant;
(b) at least one amphoteric surfactant;
(c) at least one fatty alcohol;
(d) at least one starch derivative;
(e) at least one cationic conditioning polymer;
(f) at least one amino silicone; and
(g) water.

18. The composition according to claim 17, wherein the at least one cationic surfactant is present in an amount ranging from about 0.1% to about 10% by weight, relative to the total weight of the composition.

19. The composition according to claim 17, wherein the at least one cationic surfactant is present in an amount ranging from about 1% to about 5% by weight, relative to the total weight of the composition.

20. The composition according to claim 17, wherein the at least one cationic surfactant is selected from benhentrimonim chloride, cetrimonium chloride, or mixtures thereof.

21. The composition according to claim 17, wherein the at least one amphoteric surfactant is present in an amount ranging from about 0.1% to about 10% by weight, relative to the total weight of the composition.

22. The composition according to claim 17, wherein the at least one amphoteric surfactant is present in an amount ranging from about 1% to about 5% by weight, relative to the total weight of the composition.

23. The composition according to claim 17, wherein the at least one amphoteric surfactant is selected from coco-betaine, cocamidopropylbetaine, sodium cocoamphoacetate, disodium cocoamphodiacetate, or mixtures thereof.

24. The composition according to claim 17, wherein the at least one starch derivative is present in an amount ranging from about 0.1% to about 10%, relative to the total weight of the composition.

25. The composition according to claim 17, wherein the at least one starch derivative is present in an amount ranging from about 0.5% to about 5% by weight, relative to the total weight of the composition.

26. The composition according to claim 17, wherein the at least one starch derivative is potato starch modified.

27. The composition according to claim 17, wherein the at least one cationic conditioning polymer is present in an amount ranging from about 0.01% to about 4% by weight, relative to the total weight of the composition.

28. The composition according to claim 17, wherein the at least one cationic conditioning polymer is present in an amount ranging from about 0.05% to about 2% by weight, relative to the total weight of the composition.

29. The composition according to claim 17, wherein the at least one cationic conditioning polymer is selected from polyquaternium-7, guar hydroxypropyltrimonium chloride, or mixtures thereof.

30. The composition according to claim 17, wherein the at least one amino silicone is present in an amount ranging from about 0.01% to about 5% by weight, relative to the total weight of the composition.

31. The composition according to claim 17, wherein the at least one amino silicone is present in an amount ranging from about 0.1% to about 1% by weight, relative to the total weight of the composition.

32. The composition according to claim 17, further comprising at least one cellulosic thickening agent in an amount ranging from about 0.01% to about 2% by weight, relative to the total weight of the composition.

33. A composition comprising:
(a) from about 1% to about 5% by weight of at least one cationic surfactant;
(b) from about 1% to about 5% by weight of at least one amphoteric surfactant;
(c) from about 2% to about 11% by weight of at least one fatty alcohol;
(d) from about 0.5% to about 5% by weight of at least one starch derivative;
(e) from about 0.05% to about 2% by weight of at least one cationic conditioning polymer;
(f) from about 0.1% to about 2% by weight of at least one amino silicone; and
(g) from about 70% to about 90% by weight of water;
wherein all weights being relative to the total weight of the composition.

34. The composition according to claim 33, further comprising at least one cellulosic thickening agent in an amount ranging from about 0.1% to about 0.5% by weight, relative to the total weight of the composition.

35. The composition according to claim 17, the composition having a viscosity ranging from about 180 cPs to about 400 cPs at 25° C.

36. A cosmetic treatment process for caring for and/or conditioning keratin materials, comprising:
applying onto the keratin materials a composition, the composition comprising:
(a) at least one cationic surfactant,
(b) at least one amphoteric surfactant,
(c) at least one fatty alcohol,
(d) at least one starch derivative,
(e) at least one cationic conditioning polymer,
(f) at least one amino silicone, and
(g) water; and
optionally, rinsing the keratin materials after a leave-in time.
